(19) United States
(12) Patent Application Publication
(10) Pub. No.: US 2013/0012483 A1
(43) Pub. Date: Jan. 10, 2013

(54) NOVEL PHARMACEUTICAL COMPOSITION FOR THE TREATMENT OF NEURODEGENERATIVE OR NEUROVASCULAR DISEASES

(76) Inventors: Olivier Beauchet, Avrille (FR); Cédric Annweiler, Angers (FR)

(21) Appl. No.: 13/576,673

(22) PCT Filed: Mar. 4, 2011
(86) PCT No.: PCT/EP11/53274

§ 371 Date: Sep. 26, 2012

Related U.S. Application Data
(60) Provisional application No. 61/310,789, filed on Mar. 5, 2010.

(30) Foreign Application Priority Data
Mar. 5, 2010 (EP) 10305227.0

(51) Int. Cl. A61K 31/592; A61P 25/28

(52) U.S. Cl. 514/167

(57) ABSTRACT

The present invention relates to a pharmaceutical composition comprising an NMDA receptor antagonist and vitamin D as active ingredients for the treatment of neurodegenerative or neurovascular diseases.

The present invention relates to a novel pharmaceutical composition for the treatment of neurodegenerative or neurovascular diseases.

Neurodegenerative diseases are diseases which generally result in a deterioration of the function of nerve cells, in particular neurons, or even in cell death thereof. The consequence for the patient is therefore a progressive, and in most cases irreversible, impairment of nerve functions that can result in the death of the individual affected by such diseases.

These diseases affect nervous system function, and more particularly brain function, gradually over the course of their development. This development may be more or less long, possibly ranging from a few weeks to several years.

Depending on the regions of the nervous system affected by the disease, the disorders observed may concern motivity or all or some of the cognitive functions, such as language, memory, perception or other cognitive functions.

Glutamate is the anionic form of glutamic acid which is essential for correct functioning of the nerve cells of the central nervous system, and more specifically of the brain. It is in fact one of the 20 natural α-amino acids constituting the nerve cell proteins. Moreover, it is the most widespread excitatory neurotransmitter in the central nervous system. It acts by binding to ionotropic (N-methyl-D-aspartate (NMDA), AMPA, kainate) or metabotropic post-synaptic glutamatergic membrane receptors. Glutamatergic synapses represent 50% of the synapses in the central nervous system. This glutamatergic nerve transmission has been associated, from a clinical point of view, with learning and memorising abilities.

In the event of excessive release into the synaptic cleft, glutamate is responsible for a neurotoxic excitatory message (known as neuronal excitotoxicity) which results in neuronal death. Such a dysfunction of glutamatergic neurotransmission is suspected of being involved in the physiopathology of neurodegenerative diseases such as Alzheimer's disease.

The excess glutamate released into the synaptic cleft is indeed responsible for an overexcitation of the post-synaptic glutamatergic receptors, at the forefront of which are the NMDA receptors, with as a result an excessive entry of calcium into the post-synaptic neuron, resulting in neuronal death. In vitro, the intensity of this initial stimulus induces two distinct mechanisms of neuronal death:
intraneuronal calcium influx is very large and exceeds storage capacities (situation A). In this case, excitotoxicity is immediate and results in a loss of the mitochondrial membrane potential and of energy charge and in the collapse of internal homeostasis (passive swelling of the nucleus and of the neuronal cell, cytoplasmic organelles affected, release of the intracellular and intranuclear content into the extracellular medium). It is an immediate neuronal necrosis phenomenon;
intraneuronal calcium influx is moderately excessive (situation B). In this case, excitotoxicity is delayed and linked to oxidative stress: the excessive entry of calcium into the neuron causes a cascade of events, among which are the activation of nitric oxide synthase and the synthesis of nitric oxide (NO), or the stimulation of phospholipase A2, or else calcium entry into the mitochondrion, leading to the generation of superoxide anion (O₂⁻). NO can interact with O₂⁻ to form peroxynitrite (OONO⁻). These oxidation and reduction reactions produce free radicals which induce dose-dependent neuronal damage (damage to deoxyribonucleic acid (DNA), membrane lipids by lipid peroxidation, essential cell proteins, and enzymatic inactivation). These modifications are accompanied in vitro by cell retraction, by organelle relocalisation, by chromatin condensation, by nuclear fragmentation, by the production of apoptotic bodies containing cytoplasmic and nuclear fragments: it is a delayed neuronal apoptosis phenomenon (programmed cell death in response to the toxic stimulus).

The clinical consequence of these chain reactions for glutamatergic neurons is the loss of learning and memory capacities characterizing dementia syndromes.

One of the neurodegenerative diseases most frequently encountered and diagnosed is Alzheimer's disease.

In most neurodegenerative diseases, such as, for example, Alzheimer's disease, four distinct stages are distinguished in the development of the disease:

1) the pre-dementia stage;

2) the mild stage (or first stage of dementia);

3) the moderate stage (or second stage of dementia); and

4) the severe stage (or third stage of dementia).

Alzheimer's disease usually begins with memory problems. Nevertheless, this disease can also manifest itself through the occurrence of other symptoms, such as depression, loss of functional independence, repeated falls, weight loss or else behavioural problems.

At a more advanced stage (starting from the second stage of dementia), other cognitive problems progressively appear: impairment of language, of praxis, of motivity or of communication.

There is currently no treatment which makes it possible to cure patients suffering from neurodegenerative diseases such as Alzheimer's disease, or even which makes it possible to stop the development of the disease. Some medicaments can, however, delay the development of the disease, delaying the deterioration or the loss of memory, of language or of reasoning. Among the medicaments most commonly prescribed in an attempt to slow down the development of these diseases are in particular NMDA receptor antagonists.

The NMDA receptor is an ionotropic receptor allowing transfer of electrical signals between neurons in the brain and in the spinal column. For electrical signals to pass, the NMDA receptor must be open. To remain open, an NMDA receptor must bind to glutamate and to glycine. An NMDA receptor that is bound to glycine and glutamate and has an open ion channel is called “activated”. Chemical substances that deactivate the NMDA receptor are called NMDA receptor antagonists. NMDA receptor antagonists fall into four categories:
competitive antagonists, which bind to and block the binding site of the neurotransmitter glutamate;
glycine antagonists, which bind to and block the glycine site;
noncompetitive antagonists, which inhibit NMDA receptors by binding to allosteric sites; and
uncompetitive antagonists, which block the ion channel by binding to a site within it. Examples of NMDA receptor antagonists are memantine, amantadine, riluzole and dextrometorphane.

Among the medicaments most commonly prescribed in an attempt to slow down the development of neurodegenerative diseases such as Alzheimer's disease is in particular memantine, which is a voltage-dependent, low-affinity, noncompetitive NMDA receptor antagonist. It has no immediate effect, but, after 3 to 6 months of use, the patients who receive the treatment have better cognitive functions and autonomy than the patients who received the placebo. Nevertheless, with this treatment, the decline is delayed but not treated. The efficacy of this medicament was established by means of double-blind trials versus placebo. Specifically, the low affinity and rapid kinetics of withdrawal of memantine at the level of the NMDA receptor channel preserves the physiological function of these receptors, which remain activatable by the glutamate released following depolarisation of presynaptic neurons. Nevertheless, by binding to NMDA receptors with greater affinity than Mg²⁺ magnesium ions, memantine is capable of inhibiting the prolonged influx of Ca²⁺ calcium ions. This results in protection of glutamatergic neurons, associated with avoidance of situation A mentioned above, and neuronal necrosis phenomena subsequent to the excessive and prolonged influx of calcium into the cell. Patients with Alzheimer's disease who are taking memantine are therefore in situation B discussed above, i.e. neuronal death due to immediate excitotoxicity is limited, but there is still a moderately excessive influx of calcium into the post-synaptic neuron, resulting in oxidative stress from reactive oxygen species, nitrogen and free radicals, which results in neuronal death due to apoptosis.

Thus, while NMDA receptor antagonists such as memantine allows symptomatic treatment of Alzheimer's disease, i.e. treatment which makes it possible to slow down the development of the disease, they do not allow a preventive or curative treatment of said disease that would make it possible to prevent its appearance or to treat said disease.

Vitamin D is a steroid hormone which binds to vitamin D steroid receptors (VDRs) present in neurons and glial cells of the central nervous system, including the hippocampus, the hypothalamus, the cortex or the subcortex. 1,25-OHD (active form of vitamin D) has precisely demonstrated, in vitro, neuroprotective qualities against glutamate toxicity by virtue of antioxidant effects. This detoxification action was described in 2001 on cultures of rat mesencephalic cells by Ibi et al., in an article titled Protective effects of 1 alpha,25-(OH)(2)D(3) against the neurotoxicity of glutamate and reactive oxygen species in mesencephalic culture, Neuropharmacology 2001; 40: 761-771.

In a position paper dated 2006 from Mark F. McCarty and titled Down-regulation of microglial activation may represent a practical strategy for combating neurodegenerative disorders, Medical Hypotheses (2006) 67, 251-259, hypothesis is made that Vitamin D might have an effect on certain neurodegenerative disorders such as Parkinson's disease, it being specified that experimental confirmation is still needed. In addition, it is specified that no similar hypothesis can be made for the treatment of Alzheimer's disease.

It is concluded in this publication that it will take some years before confirming the neuroprotective efficacy of various active ingredients among which memantine but that it may not be imprudent for patients in the early stages of neurogenerative disorders to use these drugs, providing that they can find a cooperative physicians. In addition, it is further specified that such patients might also be well advised to avail themselves of the nutrients and lifestyle measures cited in this publication, among which the ingestion of vitamin D.

Nevertheless, it has never been disclosed or confirmed that vitamin D or NMDA receptor antagonists would allow a preventive or curative treatment of neurodegenerative diseases, such as Alzheimer's disease, i.e. a treatment making it possible to prevent the appearance of the disease or to treat said disease, neither it has been suggested to specifically associate vitamin D with an NMDA receptor antagonist to obtain such results.

Thus, at the time of the present invention, there is no efficient treatment which cures patients suffering from neurodegenerative diseases, or even which makes it possible to stop the development thereof.

Furthermore, the neurodegenerative diseases described above may be associated with ischemic or haemorrhagic vascular damage, thus defining mixed conditions, worsening the cognitive function disorders. Apart from “mixed” damage, the vascular damage may be isolated and responsible for cognitive function disorders ranging from the pre-dementia stage to the severe dementia stage.

However, it has been found, entirely surprisingly, that the pharmaceutical combination of an NMDA receptor antagonist /vitamin D provides an effective solution to the problem of neuronal death and loss of cognitive function during pathological brain ageing, and therefore makes it possible to treat neurodegenerative diseases or to stop their development, irrespective of the stage of the disease. Furthermore, it has also been found that this combination also makes it possible to effectively prevent or treat neurovascular diseases, irrespective of the stage of the disease.

The present invention therefore relates to a pharmaceutical composition comprising two active ingredients chosen as being:
an NMDA receptor antagonist; and
vitamin D.

The pharmaceutical composition according to the invention allows the prevention and treatment of neurodegenerative diseases in human beings, irrespective of the stage of the disease, including at the pre-dementia stage. The pharmaceutical composition according to the invention also allows the prevention and treatment of neurovascular diseases in human beings.

In the context of the present invention:
“neurodegenerative disease” is intended to mean all of the neurodegenerative brain disorders, Alzheimer's disease, diseases related to Alzheimer's disease, frontotemporal dementias and related dementias, dementias associated with Parkinson's disease, Lewy body disease, mixed dementias combining a neurodegenerative condition and cerebral vascular damage, irrespective of the stage of the dementia syndrome and the age at which the disorders begin;
“neurovascular disease” is intended to mean all of the neurovascular brain disorders, and isolated neurovascular dementias, irrespective of the stage of the dementia syndrome and the age at which the disorders begin;
“pharmaceutically acceptable salt” of an active ingredient is intended to mean any salt of addition of said active ingredient with an inorganic or organic acid by the action of such an acid within an organic or aqueous solvent, such as an alcohol, a ketone, an ether or a chlorinated solvent, and which is acceptable from a pharmaceutical point of view;
“pharmaceutically acceptable derivative” of an active ingredient is intended to mean any “prodrug” or “metabolite” of said active ingredient, and also the pharmaceutically acceptable salt thereof;
“prodrug” of an active ingredient is intended to mean any compound, the bioconversion of which in the organism results in said active ingredient;
“metabolite” of an active ingredient is intended to mean any intermediate product resulting from the conversion of said active ingredient in the organism during a metabolic process;
“daily administration” is intended to mean an administration once a day or an administration once every 24 hours;
“weekly administration” is intended to mean an administration once a week or once every 7 days;
“bi-monthly administration” is intended to mean an administration twice a month or an administration once every 14 or 15 days;
“monthly administration” is intended to mean an administration once a month or an administration once every 28, 29, 30 or 31 days;
“quarterly administration” is intended to mean an administration once per trimester or an administration once every 3 months;
“bi-annual administration” is intended to mean an administration once per semester or an administration once every 6 months;
“continuous calendar” is intended to mean the continuous therapeutic treatment of a patient, in a manner which is unlimited and not sequenced or spaced out over time, i.e. without interruption of treatment;
memantine designates 3,5-dimethyltricyclo[3.3.1.1]decylamine, and its pharmaceutically acceptable salts or derivatives thereof;
amantadine designates tricyclo[3.3.1.13,7]décan-1-amine, and its pharmaceutically acceptable salts or derivatives thereof;
riluzole designates 2-amino-6-(trifluoromethoxy)benzothiazole, and its pharmaceutically acceptable salts or derivatives thereof;
dextrometorphane designates (+)-3-methoxy-17-methyl-9α,13α,14α-morphinan and its pharmaceutically acceptable salts or derivatives thereof;
vitamin D designates vitamin D₂ (ergocalciferol), vitamin D₃ (or cholecalciferol) or a mixture of the two, and its pharmaceutically acceptable salts or derivatives thereof; and
IU designates an International Unit, it being understood that 1 IU of vitamin D is equivalent to 0.025 μg of ergocalciferol/cholecalciferol.

The pharmaceutical composition according to the present invention contains an NMDA receptor antagonist associated to vitamin D. Preferably, the NMDA receptor antagonist is chosen as being memantine, amantadine, riluzole or dextrometorphane. More preferably, the NMDA receptor antagonist is chosen as being memantine.

The pharmaceutical composition according to the present invention contains the active ingredients in sufficient amount to provide the desired therapeutic effect, i.e. the treatment of neurodegenerative diseases making it possible to stop the development of the disease or make it regress in the treated patient, and also the prevention or treatment of neurovascular diseases.

Preferably, the following amounts of active ingredient are used to prepare the pharmaceutical composition according to the invention:
from 1 to 80 mg of NMDA receptor antagonist, preferably from 1 to 40 mg of NMDA receptor antagonist, more preferably from 5 to 20 mg of NMDA receptor antagonist;
from 100 to 20 000 IU, preferably from 200 to 10 000 IU of vitamin D, more preferably from 400 to 5000 IU of vitamin D, even more preferably from 600 to 1500 IU of vitamin D.

The pharmaceutical composition according to the present invention can be formulated in any galenical form necessary to its administration. In particular, as regards oral administration, the compositions according to the invention can be formulated in the form of coated or uncoated, effervescent, soluble, orodispersible, gastroresistant or modified-release tablets; of sugar-coated tablets; of hard-shell capsules (or gelatine capsules); of soft-shell capsules; of small pills; of granules; of pills; or of lozenges. As regards systemic administration, the composition according to the invention can be formulated in the form of sterile lyophilised powder for injection. The pharmaceutical compositions according to the present invention may therefore comprise, in addition to the active ingredients, any pharmaceutically acceptable formulation adjuvant known to the skilled artisan and which is necessary for the preparation of the pharmaceutical composition in the desired form.

Certain pharmaceutical compositions according to the present invention can be administered to any patient who is suffering from or who is likely to be suffering from a neurodegenerative or neurovascular disease.

Thus, the present invention also relates to a pharmaceutical composition as defined above for the preventive or curative treatment of neurodegenerative diseases in a human being. Preferably, the present invention relates to a pharmaceutical composition as defined above for the preventive or curative treatment of all the neurodegenerative brain disorders, of Alzheimer's disease, of diseases related to Alzheimer's disease, of frontotemporal dementias and related dementias, of dementias associated with Parkinson's disease, of Lewy body disease or else of mixed dementias combining a neurodegenerative condition and cerebral vascular damage, irrespective of the stage of the dementia syndrome and the age at which the disorders begin. More preferably, the present invention relates to a pharmaceutical composition as defined above for the preventive or curative treatment of one of the above diseases at a pre-dementia stage. Furthermore, the present invention also relates to a pharmaceutical composition as defined above for the preventive or curative treatment of neurovascular diseases in a human being. Preferably, the present invention relates to a pharmaceutical composition as defined above for the preventive or curative treatment of all the neurovascular brain disorders and isolated neurovascular dementias, in human beings, irrespective of the stage of the dementia syndrome and the age at which the disorders begin. More preferably, the present invention relates to a pharmaceutical composition as defined above for the preventive or curative treatment of one of the above diseases at a pre-dementia stage.

The pharmaceutical composition according to the invention may be administered at any moment during the day, preferably at the same time each day, before, during or after meals, without this having an influence on the efficacy of the treatment.

Furthermore, the pharmaceutical composition according to the invention may be administered daily.

The composition according to the present invention may be administered according to a continuous calendar.

The invention also relates to the use of a pharmaceutical composition as defined above for the preparation of a medicament useful for the preventive or curative treatment of neurodegenerative diseases in a human being. Preferably, the present invention relates to the use of a pharmaceutical composition as defined above for the preparation of a medicament useful for the preventive or curative treatment of all the neurodegenerative brain disorders, of Alzheimer's disease, of diseases related to Alzheimer's disease, of frontotemporal dementias and related dementias, of dementias associated with Parkinson's disease, of Lewy body disease or else of mixed dementias combining a neurodegenerative condition and cerebral vascular damage, irrespective of the stage of the dementia syndrome and the age at which the disorders begin. More preferably, present invention relates to the use of a pharmaceutical composition as defined above, for the preparation of a medicament useful for the preventive or curative treatment of one of the above diseases at a pre-dementia stage.

Furthermore, the present invention also relates to the use of a pharmaceutical composition as defined above, for the preparation of a medicament useful for the preventive or curative treatment of neurovascular diseases in a human being. Preferably, the present invention relates to the use of a pharmaceutical composition as defined above, for the preparation of a medicament useful for the preventive or curative treatment of neurovascular brain disorders, and isolated neurovascular dementias, in human beings, irrespective of the stage of the dementia syndrome and the age at which the disorders begin. More preferably, the present invention relates to the use of a pharmaceutical composition as defined above for the preparation of a medicament useful for the preventive or curative treatment of one of the above diseases at a pre-dementia stage.

The present invention also relates to the use of a pharmaceutical composition as defined above for the preparation of a medicament useful for the preventive or curative treatment of neurodegenerative or neurovascular diseases in a human being, said medicament being administered daily and according to a continuous calendar.

The present invention also relates to a method for the preventive or curative treatment of neurodegenerative diseases in a human being by administering a pharmaceutical composition as defined above. Preferably, the present invention relates to a method for the preventive or curative treatment of neurovascular brain disorders, of Alzheimer's disease, of diseases related to Alzheimer's disease, of frontotemporal dementias and related dementias, of dementias associated with Parkinson's disease, of Lewy body disease or else of mixed dementias combining a neurodegenerative condition and cerebral vascular damage, irrespective of the stage of the dementia syndrome and the age at which the disorders begin, by administering a pharmaceutical composition as defined above. More preferably, the present invention relates to a method for the preventive or curative treatment of one of the above diseases at a pre-dementia stage by administering a pharmaceutical composition as defined above.

Furthermore, the present invention also relates to a method for the preventive or curative treatment of neurovascular diseases in a human being by administering a pharmaceutical composition as defined above. Preferably, the present invention relates to a method for the preventive or curative treatment of neurovascular brain disorders, and isolated neurovascular dementias, in human beings, by administering a pharmaceutical composition as defined above, irrespective of the stage of the dementia syndrome and the age at which the disorders begin. More preferably, the present invention relates to a method for the preventive or curative treatment of one of the above diseases at a pre-dementia stage by administering a pharmaceutical composition as defined above.

The present invention also relates to a method for the preventive or curative treatment of neurodegenerative or neurovascular diseases in a human being by daily administration of a pharmaceutical composition as defined above according to a continuous calendar.

The two active ingredients constituting the novel treatment for neurodegenerative diseases according to the invention can be administered in the form of a unitary pharmaceutical composition comprising the two active ingredients allowing administration of said composition to the patient in a single intake.

Nevertheless, a separate administration of the constituent active ingredients of the novel treatment according to the invention can also be envisaged. Thus, the present invention also relates to a pharmaceutical product containing:
NMDA receptor antagonist; and
vitamin D;
   as a combination product (or pharmaceutical kit) for a simultaneous, separate or sequential administration.

Preferably, the NMDA receptor antagonist is chosen as being memantine, amantadine, riluzole or dextrometorphane. More preferably, the NMDA receptor antagonist is chosen as being memantine.

Preferably, the pharmaceutical product according to the present invention is used in the treatment of neurodegenerative diseases in a human being, such as all the neurodegenerative brain disorders, Alzheimer's disease, diseases related to Alzheimer's disease, frontotemporal dementias and related dementias, dementias associated with Parkinson's disease, Lewy body disease, or mixed dementias combining a neurodegenerative condition and cerebral vascular damage, irrespective of the stage of the dementia syndrome and the age at which the disorders begin.

The pharmaceutical product according to the present invention can also be used in the treatment of neurovascular diseases in a human being, such as neurovascular brain disorders, and isolated neurovascular dementias, irrespective of the stage of the dementia syndrome and the age at which the disorders begin.

The pharmaceutical product according to the present invention can also be used in the preventive or curative treatment of one of the above diseases in a human being at a pre-dementia stage.

Preferably, the following daily amounts of active ingredient are used to prepare the pharmaceutical product of the invention:
from 1 to 80 of NMDA receptor antagonist, preferably from 1 to 40 mg of NMDA receptor antagonist, more preferably from 5 to 20 mg of NMDA receptor antagonist;
from 100 to 20 000 IU of vitamin D, preferably from 200 to 10 000 IU of vitamin D, more preferably from 400 to 5000 IU of vitamin D, even more preferably from 600 to 1500 IU of vitamin D.

The pharmaceutical product according to the invention can of course be administered according to one of the administration schemes previously defined.

According to one preferred administration scheme for the pharmaceutical product according to the invention:
the unitary dosage form containing the NMDA receptor antagonist is administered daily; and
the unitary dosage form containing the vitamin D is administered weekly, bi-monthly, monthly, quarterly or bi-annually.

Of course, the amount of active ingredient contained in each unitary dosage form will be adjusted according to the frequency of administration envisaged and the daily amount of active ingredient that must be administered. By way of example, mention may be made of the unitary dosage forms containing:
from 200 to 10 000 IU of vitamin D, preferably from 400 to 5000 IU of vitamin D, more preferably from 800 to 1500 IU of vitamin D, for daily administration; and
from 1400 to 70 000 IU of vitamin D, preferably from 2800 to 35 000 IU of vitamin D, more preferably from 5600 to 10 500 IU of vitamin D, for weekly administration.

The present invention will now be illustrated in a non-limiting manner with the following examples.

### EXAMPLE

### Clinical Study of the Efficacy of the Combination Memantine+Vitamin D in Comparison to Memantine Alone and to Vitamin D Alone

1. Objective

Based on a before-after designed study, we compared the change in global cognitive performance using the Mini-Mental State Examination (MMSE) among elderly patients suffering from Alzheimer disease or related disorders (ADRD).

Three different treatments were administered to these patients:
vitamin D₃ alone,
memantine alone,
vitamin D₃ in combination with memantine.

2. Materials and Methods

2.1—Studied Population

All patients suffering from ADRD who were prescribed memantine and/or vitamin D₃ with no cerebral vasodilatators and no anticholinesterasics (i.e., donepezil, galantamine or rivastigmine) at the University Memory Center of Angers University Hospital, France and who had at least one follow-up visit, were enrolled in this study.

The clinical characteristics of these patients are summarized in Table 1 below.

Medications were reported by direct inquiry.

Criteria defined in American Psychiatric Association, Diagnostic and Statistical Manual of Mental Disorder; 4^{th} Ed. Washington, 1994 (DSM IV) were used to establish the clinical diagnosis of dementia in the absence of delirium and regardless of the length and stage of dementia.

ADRD was diagnosed according to the criteria of the NINCDS-ADRDA work group (McKhann G, Drachman D, Folstein M, Katzman R, Price D, Stadlan E M, Clinical diagnosis of Alzheimer's disease: report of the NINCDSADRDA Work Group under the auspices of Department of Health and Human Services Task Force on Alzheimer's Disease; Neurology 1984; 34: 939-944).

2.2—Primary Evaluation Criterion: Between-Visits Change in Global Cognitive Performance

The MMSE score was used to assess global cognitive performance during the study and was completed at baseline (i.e., before the drug prescription) and during the first follow-up visit. MMSE is a well-established measure of cognitive function in elderly people (see for example Folstein M F, Folstein S E, et al., Mini-Mental State: A practical method for grading the cognitive state of patients for the clinician; Journal of Psychiatric Research 1975; 12: 189-198; or Kalafat L, Hugonot-Diener L, Poitrenaud J., Standardisation et étalonnage français du<<Mini Mental State>>version Greco; Neuropsychol Rev 2003; 13: 209-236). It is a brief, practical screening test for cognitive dysfunction composed of five sections (orientation, registration, attention—calculation, recall, and language). It shows good test-retest and inter-rater reliability and performs satisfactorily against more detailed measures of cognitive function. Scores range from 30 (unimpaired) to 0 (impaired). The change in MMSE score was used to assess the change in cognitive performance after the introduction of the drug(s).

2.3—Explanatory Variable: Treatment

Subjects were prescribed either vitamin D₃ alone, memantine alone, or Vitamin D₃ in combination with memantine.

Each subject was first prescribed one of these treatments at his first visit.

Patients generally took their treatment alone. When necessary (eg, moderately-severe to severe dementia), the treatment was administered by a nurse to ensure good compliance to said treatment.

Vitamin D₃ was administered orally either daily or monthly. Dosage of vitamin D₃ ranges between 800 and 3500 IU per day.

Memantine was administered orally every day at once. Dosage ranges from 5 to 20 mg per day.

2.4—Clinical Characteristics

Age, gender, the MMSE score at baseline assessment as well as the time between two visits expressed in years (corresponding to the duration of treatment) were used as co-variables.

2.5—Statistical Analysis

The subjects' baseline characteristics were summarized using means and standard deviations or frequencies and percentages, as appropriate. First, comparisons between subjects separated into three groups corresponding to the three treatments administered (i.e., vitamin D₃ alone, memantine alone and Vitamin D₃ in combination with memantine) were performed using the Chi-square test or the Kruskal-Wallis test, as appropriate. To address the problem of multiple comparisons, analyses were completed by a post hoc Bonferonni test.

Second, multiple linear regression analyses (i.e., fully adjusted method) were performed to examine the association between the change in MMSE score (dependent variable) and the use of vitamin D₃ in combination with memantine (independent variable), after adjustment for clinical characteristics.

Three models were performed:
first, we examined the between-visits change in MMSE score taking as reference group the subjects using vitamin D₃ alone;
second, we performed the same analysis taking as reference group the subjects using memantine alone; and
third, taking as reference group the subjects using vitamin D₃ or memantine.

Analyzes were conducted separately for each of these models. P-values less than 0.05 were considered statistically significant. All statistics were performed using SPSS (version 15.0; SPSS, Inc., Chicago, Ill.).

3. Results

43 subjects (mean age 84.7±6.3 years, range 69.3−98.3 years; 65.1% women) met the inclusion criteria. No subjects were lost to follow-up. The mean MMSE score at baseline was 16.7±4.6. The mean time between 2 visits was 0.6±0.4 years.

The results of the study are reported in the following tables 1 and 2.

3.1—

TABLE 1
Characteristics and comparison of the subjects (n = 43) separated into three groups based on treatment

| | | | | P-Value* | | | |
|---|---|---|---|---|---|---|---|
| | V. D₃ | M. | V. D₃ + M. | | V. D₃ vs. | V. D₃ vs. | M. vs. |
| | (n = 17) | (n = 18) | (n = 8) | Overall | M. | V. D₃ + M. | V. D₃ + M. |
| Age, mean ± SD | 84.8 ± 4.6 | 84.0 ± 6.1 | 86.0 ± 10.2 | 0.632 | — | — | — |
| (years) | | | | | | | |
| Female, n (%) | 12 (70.6) | 12 (66.7) | 4 (50.0) | 0.592 | | | |
| MMSE score (/30 | 18.2 ± 5.0 | 16.5 ± 5.0 | 13.8 ± 2.7 | 0.094 | — | — | — |
| points), mean ± SD | | | | | | | |
| before treatment | | | | | | | |
| MMSE score (/30 | 17.7 ± 6.5 | 16.1 ± 5.1 | 17.6 ± 2.4 | 0.680 | — | — | — |
| points), mean ± SD | | | | | | | |
| after treatment | | | | | | | |
| Change in MMSE | −0.6 ± 3.1 | −0.0 ± 1.8 | 4.0 ± 3.7 | 0.019 | 0.655 | 0.011 | 0.009 |
| score^{†}, mean ± SD | | | | | | | |
| (points) | | | | | | | |
| Time between | 0.5 ± 0.3 | 0.8 ± 0.5 | 0.5 ± 0.3 | 0.155 | — | — | — |
| visits, mean ± SD | | | | | | | |
| (years) | | | | | | | |

V. D₃: vitamin D₃
M: memantine
MMSE: Folstein Mini-Mental State Examination
*Comparison based on the Kruskal-Wallis with Bonferonni corrections, or the Chi-square test, as appropriate
^{†}Calculated from the formula “MMSE score after treatment − MMSE score before treatment”
P significant (i.e., <0.05) indicated in bold.

3.2—

TABLE 2
Multiple linear regression models* (fully adjusted models) showing the association of the
between-visits change in MMSE score† (dependent variable) with the use of vitamin D3
together with memantine (independent variable), adjusted for clinical characteristics (n = 43)

| | Model 1* (n = 25) | | | Model 2* (n = 26) | | | Model 3* (n = 43) | | |
|---|---|---|---|---|---|---|---|---|---|
| | β | 95% CI | P-value | β | 95% CI | P-value | β | 95% CI | P-value |
| V. D₃ + M. | 4.91 | [0.90; 8.92] | 0.020 | 3.20 | [0.53; 5.86] | 0.021 | 3.63 | [0.52; 6.74] | 0.023 |
| Age | 0.06 | [−0.21; 0.33] | 0.665 | 0.08 | [−0.07; 0.23] | 0.281 | 0.11 | [−0.06; 0.29] | 0.190 |
| Female | −0.08 | [−3.88; 3.72] | 0.966 | −0.99 | [−3.12; 1.21] | 0.358 | −0.38 | [−2.57; 1.82] | 0.729 |
| MMSE score before | 0.06 | [−0.36; 0.49] | 0.753 | −0.12 | [−0.40; 0.15] | 0.347 | −0.06 | [−0.33; 0.21] | 0.653 |
| treatment | | | | | | | | | |
| Time between visits | −1.24 | [−7.66; 5.18] | 0.688 | −0.70 | [−3.35; 1.95] | 0.586 | −0.78 | [−3.70; 2.14] | 0.591 |

V. D₃: vitamin D₃
M.: memantine
CI = confident interval
MMSE: Folstein Mini-Mental State Examination
β: Coefficient of regression beta corresponding to a between-visits change in MMSE score expressed in points
*separated models:
Model 1: versus subjects using vitamin D3 only;
Model 2: versus subjects using memantine only;
Model 3: versus subjects using vitamin D3 or memantine
†Calculated from the formula “MMSE score after treatment − MMSE score before treatment”
Coefficient of regression β significant (i.e., P < 0.05) indicated in bold

3.3—Comments

As reported in Table 1:
17 subjects received vitamin D₃ only (39.5%),
18 subjects received memantine only (41.9%), and
8 subjects received vitamin D₃ in combination with memantine (18.6%).

The three groups were comparable at baseline with no significant difference regarding the age (P=0.632), gender (P=0.592), and baseline MMSE score (P=0.094). The time between two visits did not differ between groups (P=0.155) (Table 1). The only significant between-group difference was the change in MMSE score after oral treatment (P=0.019). The change in MMSE score of 4.0±3.7 points among subjects using vitamin D₃ in combination with memantine was higher than among subjects using vitamin D₃ alone (P=0.009) or memantine alone (P=0.011). There was also no difference between these last two groups (P=0.655): subjects using vitamin D₃ alone lost 0.6±3.1 points in MMSE score on average, while the group under memantine was stabilized (change in MMSE score of 0.0±1.8 point).

Table 2 shows multiple linear regressions for between-visits change in MMSE score, with the use of vitamin D₃ in combination with memantine and the other clinical characteristics as explanatory variables. After adjustment, using vitamin D₃ in combination with memantine was positively associated with the change in MMSE score while taking as reference group the subjects using vitamin D₃ alone (adjusted β=4.91, P=0.020) (Model 1). In addition, Model 2 showed that using vitamin D₃ in combination with memantine was positively associated with the change in MMSE score while taking as reference group the subjects using memantine alone (adjusted β=3.20, P=0.021). Finally, using vitamin D₃ and memantine was also positively associated with the change in MMSE score while taking as reference group the subjects using vitamin D₃ or memantine (adjusted β=3.63, P=0.023) (Model 3).

4. Conclusion

These results demonstrate that administering vitamin D₃ in combination with memantine allows a significant gain of cognitive performance for patients thus treated in comparison to patients treated by vitamin D₃ or memantine alone. This gain was independent of the gender, the age and the baseline cognitive performance. In addition, the gain was high and significant against the consumption of the two molecules taken separately, and even against taking one or the other, which highlighted the synergistic effect of the combination of vitamin D₃ and memantine.

A synergy of action between vitamin D₃ and memantine is hereby demonstrated allowing a reverse effect on cognition and brain disorders compared to the use of memantine or vitamin D alone allowing at best a stabilisation of cognitive performance.

1. Pharmaceutical composition comprising two active ingredients chosen as being:
an NMDA receptor antagonist; and
vitamin D.

2. The pharmaceutical composition according to claim 1, wherein the NMDA receptor antagonist is chosen as being memantine, amantadine, riluzole or dextrometorphane.

3. The pharmaceutical composition according to claim 2, wherein the NMDA antagonist receptor is memantine.

4. The pharmaceutical composition according to claim 1, comprising from 1 mg to 80 mg of NMDA receptor antagonist.

5. The pharmaceutical composition according to claim 1, further comprising from 100 IU to 20 000 IU of vitamin D.

6. The pharmaceutical composition according to claim 1, for the preventive or curative treatment of neurodegenerative diseases.

7. The pharmaceutical composition according to claim 6, wherein the neurodegenerative disease is chosen from all the neurodegenerative brain disorders, Alzheimer's disease, diseases related to Alzheimer's disease, frontotemporal dementias and related dementias, dementias associated with Parkinson's disease, Lewy body disease, and mixed dementias combining a neurodegenerative condition and cerebral vascular damage.

8. The pharmaceutical composition according to claim 1, for the preventive or curative treatment of neurovascular diseases.

9. The pharmaceutical composition according to claim 6, wherein the disease is at the pre-dementia stage.

10. The pharmaceutical composition according to claim 1, said pharmaceutical composition being administered daily and according to a continuous calendar.

11. Pharmaceutical product containing:
an NMDA receptor antagonist as defined in claim 1; and
vitamin D;
as a combination product for a simultaneous, separate or sequential administration.

12. The pharmaceutical product according to claim 11 for the treatment of neurodegenerative diseases in a human being.

13. The pharmaceutical product according to claim 12, wherein the neurodegenerative disease is chosen from all the neurodegenerative brain disorders, Alzheimer's disease, diseases related to Alzheimer's disease, frontotemporal dementias and related dementias, dementias associated with Parkinson's disease, Lewy body disease, and mixed dementias combining a neurodegenerative condition and cerebral vascular damage.

14. The pharmaceutical product according to claim 11 for the treatment of neurovascular diseases in a human being.
